(19) United States
(12) Patent Application Publication
(10) Pub. No.: US 2004/0072287 A1
(43) Pub. Date: Apr. 15, 2004

(54) Cell lines expressing a mch receptor

(76) Inventors: Carina Tan, Rahway, NJ (US); Margarita Nossoughi, Rahway, NJ (US); Shigeru Tokita, Tokyo (JP)

Correspondence Address: MERCK AND CO INC, P O BOX 2000, RAHWAY, NJ, 070650907

(21) Appl. No.: 10/415,508

(22) PCT Filed: Oct. 26, 2001
(86) PCT No.: PCT/US01/47027

(51) Int. Cl. C12P 21/02; C12N 5/08; C07K 14/705

(52) U.S. Cl. 435/69.1; 435/320.1; 435/368; 530/350; 536/23.5

(57) ABSTRACT

The present invention features neuroblastoma and skin cell carcinoma cell lines functionally expressing MCHR1 and the use of such cells to measure MCHR1 activity. Functional expression is preferably achieved in a neuroblastoma or skin cell carcinoma using a recombinant gene expressing MCHR1. The presence of a recombinant MCHR1 gene increases the level of MCHR1 expression facilitating the production and detection of MCHR1 activity.

### CROSS-REFERENCE TO RELATED APPLICATIONS

The present application claims priority to provisional application U.S. Ser. No. 60/244,700, filed Oct. 31, 2000, hereby incorporated by reference herein.

### BACKGROUND OF THE INVENTION

The references cited herein are not admitted to be prior art to the claimed invention.

Neuropeptides present in the hypothalamus play a major role in mediating the control of body weight. (Flier, et al., 1998. Cell, 92, 437440.) Melanin-concentrating hormone (MCH) is a cyclic 19-amino acid neuropeptide synthesized as part of a larger pre-prohormone precursor in the hypothalamus which also encodes neuropeptides NEI and NGE. (Nahon, et al., 1990. Mol. Endocrinol. 4, 632-637.) MCH was first identified in salmon pituitary, and in fish MCH affects melanin aggregation thus affecting skin pigmentation. In trout and in eels MCH has also been shown to be involved-in stress induced or CRF-stimulated ACTH release. (Kawauchi, et al., 1983. Nature 305, 321-323.)

In humans two genes encoding MCH have been identified that are expressed in the brain. (Breton, et al., 1993. Mol. Brain Res. 18, 297-310.) In mammals MCH has been localized primarily to neuronal cell bodies of the hypothalamus which are implicated in the control of food intake, including perikarya of the lateral hypothalamus and zona inertia. (Knigge, et al., 1996. Peptides 17, 1063-1073.)

Pharmacological and genetic evidence suggest that the primary mode of MCH action is to promote feeding (orexigenic). MCH mRNA is up regulated in fasted mice and rats, in the ob/ob mouse and in mice with targeted disruption in the gene for neuropeptide Y (NPY). (Qu, et al., 1996. Nature 380, 243-247, and Erickson, et al., 1996. Nature 381, 415-418.) Injection of MCH centrally (ICV) stimulates food intake and MCH antagonizes the hypophagic effects seen with α melanocyte stimulating hormone (αMSH). (Qu, et al., 1996. Nature 380, 243-247.) MCH deficient mice are lean, hypophagic and have increased metabolic rate. (Shimada, et al., 1998. Nature 396, 670-673.)

MCH action is not limited to modulation of food intake as effects on the hypothalamic-pituitary-axis have been reported. (Nahon, 1994. Critical Rev. in Neurobiol. 8, 221-262.) MCH may be involved in the body response to stress as MCH can modulate the stress-induced release of CRF from the hypothalamus and ACTH from the pituitary. In addition, MCH neuronal systems may be involved in reproductive or maternal function.

Several references describe a receptor that is indicated to bind MCH (human MCHR1). (Chambers, et al., 1999. Nature 400, 261-265; Saito, et al., 1999. Nature 400, 265-269; Bäichner, et al., 1999. FEBS Letters 457, 522-524; Shimomura, et al., 1999. Biochemical and Biophysical Research Communications 261, 622-626; and Lembo, et al., 1999. Nat. Cell Biol. 1, 267-27 1.)

### SUMMARY OF THE INVENTION

The present invention features neuroblastoma and skin cell carcinoma cell lines functionally expressing MCHR1 and the use of such cells to measure MCHR1 activity. Functional expression is preferably achieved in a neuroblastoma or skin cell carcinoma using a recombinant gene expressing MCHR1. The presence of a recombinant MCHR1 gene increases the level of MCHR1 expression facilitating the production and detection of MCHR1 activity.

Thus, a first aspect of the present invention describes a neuroblastoma or skin cell carcinoma comprising a recombinant MCHR1 gene that expresses functional MCHR1. Functional MCHR1 produces a detectable signal upon MCH stimulation. The recombinant MCHR1 gene may be part of a genome or may be present outside of the genome.

An MCHR1 gene contains nucleic acid encoding for MCHR1 and regulatory elements needed for functional expression. Examples of regulatory elements useful for functional expression include a promoter, a terminator, a ribosome binding site, and a polyadenylation region. The nucleic acid encoding for MCHR1 can be contiguous or may contain one or more introns.

A recombinant MCHR1 gene encodes for MCHR1 and contains one or more regions not naturally associated with each other. Examples of recombinant MCHR1 genes include those containing a human nucleic acid sequence encoding for MCHR1 present with a regulatory sequence not naturally associated with the encoding nucleic acid; and those containing a non-naturally occurring encoding region. A non-naturally encoding region contains one or more combinations of nucleotides not present in the naturally occurring encoding nucleic acid. Recombinant genes can be produced with, or without, intron(s).

Another aspect of the present invention describes a neuroblastoma or skin cell carcinoma having increased MCHR1 expression produced by a process comprising the step of coupling endogenous nucleic acid encoding for MCHR1 to an exogenous promoter. The process results in the production of a recombinant MCHR1 gene having the same chromosomal location as the native MCHR1 gene.

Another aspect of the present invention describes a method of measuring the ability of a compound to affect MCHR1 activity. The method comprises the steps of: (a) providing a compound to a neuroblastoma or skin cell carcinoma expressing MCHR1; and (b) measuring MCHR1 activity.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### DETAILED DESCRIPTION OF THE INVENTION

Human neuroblastoma and skin cell carcinoma cell lines able to produce MCHR1 transcripts and to provide a suitable environment for measuring MCHR1 activity are identified herein. Human cell lines expressing MCHR1 provide a human cellular environment that naturally expresses MCHR1.

Cells expressing functional MCHR1 provide a system for screening for compounds active at MCHR1, measuring the effect of a compound at MCHR1, and measuring the effect of MCHR1 activity. MCHR1 expression in a neuroblastoma or skin cell carcinoma is preferably increased using a recombinant MCHR1 gene that either makes use of endogenous nucleic acid encoding for MCHR1 or provides exogenous nucleic acid encoding MCHR1.

Compounds modulating MCHR1 activity have a variety of different uses including utility as a tool to further study MCHR1 activity and as an agent to achieve a beneficial effect in a patient. Beneficial effects of modulating MCHR1 activity include one or more of the following: weight loss, weight gain, cancer treatment (e.g., colon or breast), pain reduction, diabetes treatment, stress reduction and sexual dysfunction treatment.

Modulating MCHR1 activity includes evoking a response at the receptor and altering a response evoked by a MCHR1 agonist or antagonist. Generally, MCH receptor antagonists and allosteric modulators negatively affecting activity may be used to achieve weight loss, treat cancer (e.g., colon or breast), reduce pain, reduce stress or treat sexual dysfunction; and MCH receptor agonists and allosteric modulators positively affecting activity may be used to produce a weight gain.

A patient is a mammal, preferably a human. Reference to patient does not necessarily indicate the presence of a disease or disorder. The term patient includes subjects treated prophylactically and subjects afflicted with a disease or disorder.

Preferably, MCHR1 activity is modulated to treat diabetes, to obtain a weight loss, or to obtain a weight gain. Diabetes mellitus can be treated by, for example, one or both of the following: enhancing glucose tolerance and decreasing insulin resistance.

Excessive weight is a contributing factor to different diseases including hypertension, diabetes, dyslipidemias, cardiovascular disease, gall stones, osteoarthritis and certain forms of cancers. Bringing about a weight loss can be used, for example, to reduce the likelihood of such diseases and as part of a treatment for such diseases. Weight reduction can be achieved by, for example, one or more of the following: reducing appetite, increasing metabolic rate, reducing fat intake or reducing carbohydrate craving.

Increasing weight is particularly useful for a patient having a disease or disorder, or under going a treatment, accompanied by weight loss. Examples of diseases or disorders accompanied by weight loss include anorexia, AIDS, wasting, cachexia, and frail elderly. Examples of treatments accompanied by weight loss include chemotherapy and radiation therapy.

### MCHR1

MCHR1 employed in the present invention includes naturally occurring human MCHR1 having the amino acid sequence provided by SEQ. ID. NO. 1 and variants thereof. Variants of MCHR1 have a substantially identical amino acid sequence as SEQ. ID. NO. 1 and have MCH receptor activity. Examples of SEQ. ID. NO. 1 variants include naturally occurring allelic variants and artificially produced mutants.

SEQ. ID. NO. 1 and the naturally occurring encoding nucleic acid sequence were initially identified as the somatostatin-like receptor “SLC-1.” (Lakaye, et al., 1998. Biochimica et Biophysica ACTA 1401:216-220.) Subsequently, SLC-1 was shown to be MCHR1. cDNA and genomic sequences encoding for MCHR1 are provided by SEQ. ID. NOs. 2 and 3.

In general, SEQ. ID. NO. 1 variants have a sequence identity of at least about 90%, preferably, at least about 95%, with SEQ. ID. NO. 1; and/or contain 1, 2, 3, 4, 5, 6, 7, 8, 9, or 10 amino acid modifications from SEQ. ID. NO. 1. Amino acid modifications are additions, deletions, and substitutions.

Sequence similarity for polypeptides can be determined by BLAST. (Altschul, et al., 1997. Nucleic Acids Res. 25, 3389-3402, hereby incorporated by reference herein.) In one embodiment, sequence similarity is determined using tBLASTn search program with the following parameters: MATRIX:BLOSUM62, PER RESIDUE GAP COST: 11, and Lambda ratio: 1.

Artificial variants of MCHR1 can be produced in a cell by introducing nucleic acid encoding for the variant. Nucleic acid sequences encoding for variants can be obtained by altering the nucleic acid sequence encoding for SEQ. ID. NO. 1. The translation of a particular codon into a particular amino acid is well known in the art (see, e.g., Lewin, GENES IV, p. 119, Oxford University Press, 1990):

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons WUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons WUA, WUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Changes to SEQ. ID. NO. 1 to produce functional variants may be empirically determined. Techniques for measuring MCH receptor activity are well known in the art.

One method of producing functional variants of SEQ. ID. NO. 1 expected to retain some MCH receptor activity takes into account differences in amino acid R groups. An R group effects different properties of an amino acid such as physical size, charge, and hydrophobicity. Amino acids can be divided into different groups as follows: neutral and hydrophobic (alanine, valine, leucine, isoleucine, proline, tryptophan, phenylalanine, and methionine); neutral and polar (glycine, serine, threonine, tyrosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolor amino acids in the interior of a polypeptide then glutamate because of its long aliphatic side chain. (See, Ausubel, Current Protocols in Molecular Biology, John Wiley, 1987-1998, Supplement 33 Appendix IC.)

### Recombinant MCHR1 Gene

A recombinant MCHR1 gene can be used to increase the level of MCHR1 expression in a neuroblastoma or skin cell carcinoma thereby facilitating the production and detection of MCHR1 activity. Methods for producing a recombinant MCHR1 gene include those altering the endogenous MCHR1 gene and those introducing an MCHR1 gene or coding region into a host cell.

Alterations of an endogenous MCHR1 gene producing a recombinant gene include the use of regulatory elements such as a promoter or enhancer not naturally associated with the MCHR1 coding region; and using a non-naturally encoding region containing one or more combinations of nucleotides not present in the naturally occurring encoding nucleic acid. Examples of exogenous promoters include the human cytomegalovirus promoter (“CMV”), α-MHC promoter, PrP (prion promoter), potent neuronal promoter and Thy-1 promoter.

Non-naturally occurring encoding regions can be produced based on the degeneracy of the genetic code. If desired, the nucleic acid encoding for MCHR1 can be altered based on the genetic code to adjust codon frequency.

Techniques that can be used for creating a recombinant chromosomal gene are well known in the art. (See Ausubel, Current Protocols in Molecular Biology, John Wiley, 1987-1998). Exogenous nucleic acid can be targeted to the MCHR1 gene using homologous recombination targeting sequences. Homologous recombination targeting sequences for insertion into the MCHR1 gene include coding and non-coding regions.

An exogenous promoter, such as the CMV promoter, can be functionally coupled to MCHR1 nucleic acid using standard techniques. For example, the GOTO MCHR1 genomic sequence can be cloned into a plasmid vector. The MCHR1 promoter region (2-3 kb) upstream of coding sequence can be replaced by a CMV promoter cassette containing a loxP-neomycin-loxP gene. The resulting promoter-exchange vector would have sufficient MCHR1 genomic sequences flanking the CMV promoter cassette for homologous recombination. The vector can then be electroporated into the GOTO cell. Neomycin resistant clones can be selected and verified by genomic Southern analysis for successful promoter exchange. The neomycin gene can be removed by loxp mediated recombination to reduce its possible interference with promoter activity.

Introducing an MCHR1 gene or coding region into a host can be achieved by inserting a MCHR1 coding region or gene into the host genome or through the use of an independently replicating vector. Techniques for inserting nucleic acid into the host genome include those targeting and selecting for insertion in a particular region and those involving random insertion.

### Functional Assays

Techniques for measuring MCHR1 activity include detecting a change in the intracellular conformation of MCHR1, measuring G-protein activity, and measuring the level of intracellular messengers. Assays measuring different G-protein activities, such as Gi, Gs, and Gq can be carried out using techniques that are well known in the art. MCHR1 activity is preferably assayed for by measuring either Gi or Gq activity.

Gi and Gs activity can be measured using techniques such as a melonaphore assay, assaying cAMP production, assaying inhibition of cAMP accumulation, intracellular acidification, and assaying 35S-GTP binding. cAMP can be measured using different techniques such as a radioimmunoassay and indirectly by cAMP responsive gene reporter proteins.

Gq and Gi activity can be measured using techniques such as those detecting intracellular Ca²⁺ and intracellular acidification. Examples of techniques well known in the art that can be employed to measure Ca²⁺ include the use of dyes such as Fura-2 and the use of Ca²⁺-bioluminescent sensitive reporter proteins such as aequorin. (Button, et al., 1993. Cell Calcium 14, 663-671, and Feighner, et al., 1999. Science 284, 2184-2188, both of which are hereby incorporated by reference herein.)

Functional assays can be performed using individual compounds or preparations containing different compounds. A preparation containing different compounds where one or more compounds affect MCHR1 activity can be divided into smaller groups of compounds to identify the compound(s) affecting MCHR1 activity. In an embodiment of the present invention a test preparation containing at least 10 compounds is used in a functional assay.

### MCHR1 Expressing Cell Lines

Human cell lines able to express MCHR1 transcripts include neuroblastoma and skin cell carcinoma. The ability of different neuroblastoma cell lines and a squamous cell carcinoma is illustrated in the Examples provided below. The squamous cell carcinoma provides an example of a skin cell carcinoma. In different embodiments of the present invention concerning a skin cell carcinoma, the skin cell carcinoma is a squamous cell carcinoma or a kertinocyte cell carcinoma.

The ability of different neuroblastoma cells lines and a skin cell carcinoma to express MCHR1 transcripts points to these types of cell lines as containing members able to express MCHR1. Additional neuroblastoma and skin cell carcinoma cell lines able to express MCHR1 transcripts can be identified using routine experimentation, for example, by measuring the ability of neuroblastoma and skin cell carcinoma cell lines present in depositories such as American Type Culture Collection (Virginia, U.S.) and Health Science Research Resources Bank (Osaka, Japan) to express MCHR1 transcripts.

### Modulating MCHR1 Activity

Using the present application as a guide compounds able to modulate MCHR1 activity can be obtained and used as a research tool to further explore the affects of MCHR1 activation or as a therapeutic to achieve a beneficial effect in a patient. Beneficial effects can be obtained, for example, by using a compound active at MCHR1 to achieve one or more of the following: weight loss, weight gain, treat cancer (e.g., colon or breast), reduce pain, treat diabetes, reduce stress or teat sexual dysfunction.

Altering weight is particularly useful for gaining weight in an under weight patient or losing weight in an over weight patient. In addition, for example, farm animals can be treated to gain weight. Under weight patients include those having a body weight about 10% or less, 20% or less, or 30% or less, than the lower end of a “normal” weight range or Body Mass Index (“BMI”). Over weight patients include those having a body weight about 10% or more, 20% or more, 30% or more, or 50% or more, than the upper end of a “normal” weight range or BMI. “Normal” weight ranges are well known in the art and take into account factors such as a patient age, height, and body type.

BMI measures your height/weight ratio. It is determined by calculating weight in kilograms divided by the square of height in meters. The BMI “normal” range is 19-22.

MCHR1 modulating compounds can be provided in kit. Such a kit typically contains an active compound in dosage form for administration. A dosage form contains a sufficient amount of active compound such that a beneficial effect can be obtained when administered to a patient during regular intervals, such as 1 to 6 times a day, during the course of 1 or more days. Preferably, a kit contains instructions indicating the use of the dosage form to achieve a beneficial effect and the amount of dosage form to be taken over a specified time period.

### Dosing For Therapeutic Applications

Guidelines for pharmaceutical administration in general are provided in, for example, Reminington's Pharmaceutical Sciences 18^{th} Edition, Ed. Gennaro, Mack Publishing, 1990, and Modern Pharmaceutics 2^{nd} Edition, Eds. Banker and Rhodes, Marcel Dekker, Inc., 1990, both of which are hereby incorporated by reference herein.

MCHR1 active compounds having appropriate functional groups can be prepared as acid or base salts. Pharmaceutically acceptable salts (in the form of water- or oil-soluble or dispersible products) include conventional non-toxic salts or the quaternary ammonium salts that are formed, e.g., from inorganic or organic acids or bases. Examples of such salts include acid addition salts such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate; and base salts such as ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine.

MCHR1 active compounds can be administered using different routes including oral, nasal, by injection, and transmucosally. Active ingredients to be administered orally as a suspension can be prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate release tablets, these compositions may contain microcrystalline cellulose, dicalcium phosphate, starch, magnesium stearate and lactose and/or other excipients, binders, extenders, disintegrants, diluents and lubricants.

When administered by nasal aerosol or inhalation, compositions can be prepared according to techniques well known in the art of pharmaceutical formulation. Examples of formulation components include solutions in saline, employing benzyl alcohol or other suitable preservatives, absorption promoters to enhance bioavailability, fluorocarbons, and/or other solubilizing or dispersing agents.

The compounds may also be administered in intravenous (both bolus and infusion), intraperitoneal, subcutaneous, topical with or without occlusion, or intramuscular form. When administered by injection, the injectable solutions or suspensions may be formulated using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, compositions may be prepared by mixing the drug with a suitable non-irritating excipient, such as cocoa butter, synthetic glyceride esters or polyethylene glycols, which are solid at ordinary temperatures, but liquidify and/or dissolve in the rectal cavity to release the drug.

Suitable dosing regimens for the therapeutic applications can be selected taking into account factors well known in the art including age, weight, sex and medical condition of the patient; the severity of the condition to be treated; the route of administration; the renal and hepatic function of the patient; and the particular compound employed.

Optimal precision in achieving concentrations of drug within the range that yields efficacy without toxicity requires a regimen based on the kinetics of the drug's availability to target sites. This involves a consideration of the distribution, equilibrium, and elimination of a drug. The daily dose for a patient is expected to be between 0.01 and 1,000 mg per adult patient per day.

### EXAMPLES

Examples are provided below to further illustrate different features of the present invention. The examples also illustrate useful methodology for practicing the invention. These examples do not limit the claimed invention.

### Example 1

### MCHR1 Expression in Different Cell Lines

RT-PCR experiments were performed to determine whether different human cell lines expressed mRNA for the MCHR1. Purified poly (A)+mRNA was isolated from cultured cells using an oligo-dT kit (Poly (A) Pure, Ambion, Austin, Tex.). RT-PCR using ˜1 μg of isolated mRNA was performed using Superscript II reverse transcriptase (Life Technologies, Gaithersberg, Md.) essentially following the manufacturer's instructions. PCR cycling conditions were: 94° C. for 1 minute (one cycle), 94° C. for 30 seconds then 72° C. for 4 minutes (four cycles); 94° C. for 30 seconds then 70° C. for 4 minutes (four cycles); 94° C. for 30 seconds then 68° C. for 4 minutes (25 cycles); and 68° C. for 10 minutes (one cycle). PCR primers were chosen based on the human MCHR1 DNA sequence to flank the mRNA splice junction on either side of the single intron between exon 1 and exon 2. Forward sense primers had the following sequences: SEQ. ID. NO. 4: ATGGACCTGGAAGCCTCGCTGCTG, SEQ. ID. NO. 5: GCCAGCAACACCTCTGATGGC, and SEQ. ID. NO. 6: GGCCCCGATAACCTCACTTCGGC. Reverse (anti sense) primers had the following sequences: SEQ. ID. NO. 7: GAGGAGATCTACTACCGAGAGG, SEQ. ID. NO. 8: GCCCATGAGCTGGTGGATCATG and SEQ. ID. NO. 9: GTGGACAGTGGCCAGGTAGAGGTC.

Amplified products were electrophoresed on an agarose gel, and Southern blotted with a human MCHR1 radiolabeled probe. The probe was random prime labeled with ³²P. Post-hybridization washing stringency was at 65° C., 1×SSC, after which the filters were dried and exposed to X-ray film for 3 hours at −70° C. The results are shown in Table 1.

TABLE 1

| Cell Line | ATCC No. | Type | RT-PCR Results |
|---|---|---|---|
| HGT-1 | | Gastric carcinoma | − |
| H4 | | Neuroglioma | − |
| TE671 | | Medulloblastoma | − |
| SK-N-BE2 | CRL-2271 | Neuroblastoma | + |
| T-98G | | Glioblastoma | − |
| U-87MG | | Glioblastoma | − |
| CCF-DTTG | | Astrocytoma | − |
| MC-IXC | | Neuroblastoma | − |
| SMS-KAN | | Neuroblastoma | − |
| SMS-MSN | | Neuroblastoma | − |
| CHP-212 | CRL-2273 | Neuroblastoma | + |
| CHP-243 | CRL-2272 | Neuroblastoma | + |
| IMR32 | | Neuroblastoma | − |
| GT1-7 | | Hypothalamus (SV- | − |
| | | 40 immortalized) | |
| SH-SY5Y | CRL-2266 | Neuroblastoma | + |
| SCC-25 | CRL-1628 | Squamous cell | + |
| | | carcinoma | |
| GOTO 1 | 1 | Adrenal | + |
| | | Neuroblastoma | |

1 JCRB0612, 11, obtained from Health Science Research Resources Bank (Osaka, Japan), see Sekiguchi, et al., 1979. Japan J. Exp. Med. 49, 67-83).

Neuroblastoma cells lines GOTO, CHP-212, CHP-243, SK-N-BE(2), and SH-SY5Y were found to produce mRNA encoding for human MCHR1. In addition, the squamous cell carcinoma cell line SCC-25 was found to produce mRNA encoding for human MCHR1.

### Example 2

### MCH Receptor Activity in Neurobastoma Cells Lines

To assess whether neuroblastoma cells provide an environment for functional MCHR1, MCH receptor activity was measured using GOTO cells employing an aequorin bioluminescence assay. The aequorin bioluminescence assay can be used to measure the activity of G protein-coupled receptors that couple through the Ga protein subunit family consisting of Gq, G11, and Gi leading to the activation of phospholipase C, mobilization of intracellular calcium and activation of protein kinase C. Measurement of functional MCHR1 expression in GOTO cells transiently expressing aequorin was performed using a Luminoskan RT luminometer (Labsystems Inc., Gaithersburg, Md.) controlled by custom software written for a Macintosh PowerPC 6100.

GOTO cells (1.2×10⁷ cells plated 18 hours before transfection in a T75 flask) were transfected with human MCHR1 plasmid DNA and aequorin cDNA using the Lipofectamine 2000 procedure (Life Technologies, Gaithersburg, Md.). Human MCHR1 plasmid DNA contained the open reading frame cDNA (SEQ. ID. NO. 2) encoding the human MCHR1 receptor inserted in the mammalian expression vector pcDNA-3 (Invitrogen, Carlsbad, Calif.). An aequorin expressing plasmid contained the cDNA for aequorin (Button, et al., 1993. Cell Calcium 14, 663-671), inserted in pcDNA-3. Following approximately 40 hours of expression the apo-aequorin in the cells was charged for 1 hour with coelenterazine (10 μM) under reducing conditions (300 μM reduced glutathione) in ECB buffer (140 mM NaCl, 20 mM KCl, 20 mM HEPES-NaOH [pH=7.4], 5 mM glucose, 1 mM MgCl₂, 1 mM CaCl₂, 0.1 mg/ml bovine serum albumin). The cells were harvested, washed once in ECB medium and resuspended to 500,000 cells/ml or 1,000,000cells/ml.

100 μl of MCH or, for control responses, lysophosphatidic acid were injected into a cell suspension (corresponding to 5×10⁴ cells or 100,000 cells). Lysophosphatidic acid triggers native edg receptors coupled to PLC activation present on GOTO cells. The integrated light emission was recorded over 30 seconds, in 0.5 second units. 20 μL of lysis buffer (0.1% final Triton X-100 concentration) was then injected and the integrated light emission recorded over 10 seconds, in 0.5 second units. The “fractional response” values for each well were calculated by taking the ratio of the integrated response to the initial challenge to the total integrated luminescence including the Triton X-100 lysis response. The results are shown in Table 2.

TABLE 2

| Transfected | Buffer | LPA | MCH | MCH |
|---|---|---|---|---|
| cDNA | (ECB) | (1 μM) | (1 μM) | (1 μM) |
| No Aeg | 0 | 0 | 0 | 0 |
| AEQ | 2.7 | 5.24 | 1.9 | 3.43 |
| AEQ + | 2.76 | 6.83 | 26.4 | 26.6 |
| MCHR 1 | | | | |

The results are in bioluminescence (cps). “No Aeq” indicates the absence of plasmids encoding for aequorin and MCHR1. “AEQ” indicates the presence of a plasmid encoding for aequorin, and the absence of a plasmid encoding for MCHR1. “AEQ + MCHR1” indicates the presence of plasmids encoding for aequorin and MCHR1.

Transfection of the reporter gene aequorin into GOTO cells permitted the detection of functional MCHR1 when co-transfected with a cDNA encoding the human MCHR1 using 1 μM MCH to evoke a bioluminescent response. This observation indicates that neuroblastoma cells expressing MCHR1 are appropriate host cells for expressing the MCHR1 gene. In the absence of exogenous MCHR1, no signal over background (buffer injection only, ECB) could be observed suggesting that the level of MCHR1 naturally present in GOTO cells is insufficient to permit detection using the employed conditions. A control response evoked by the application of 1 μM lysophosphatic acid (LPA) suggests the presence of an edg receptor (Im, et al., 2000. J. Biol. Chem. 275, 14281-14286), on GOTO cells linked to calcium mobilization.

Other embodiments are within the following claims. While several embodiments have been shown and described, various modifications may be made without departing from the spirit and scope of the present invention.

What is claimed is:

1. A neuroblastoma cell or a skin cell carcinoma comprising a recombinant melanin-concentrating hormone receptor 1 (MCHR1) gene that expresses functional MCHR1.

2. The cell of claim 1, wherein said cell is a human neuroblastoma cell.

3. The neuroblastoma cell of claim 2, wherein said recombinant MCHR1 gene is present in the neuroblastoma cell genome and comprises endogenous nucleic acid encoding for MCHR1 transcriptionally coupled to an exogenous promoter.

4. The neuroblastoma cell of claim 3, wherein said exogenous promoter is a CMV promoter.

5. The neuroblastoma of claim 3, wherein said cell further comprises a recombinant gene encoding for aequorin.

6. The neuroblastoma cell of claim 3, wherein said neuroblastoma cell is selected from the group consisting of: GOTO, CHP-212, CHP-243, SK-N-BE(2), and SH-SY5Y.

7. The cell of claim 1, wherein said cell is SCC-25.

8. A neuroblastoma cell or skin cell carcinoma having increased MCHR1 expression produced by a process comprising the step of coupling endogenous nucleic acid encoding for MCHR1 to an exogenous promoter.

9. The cell of claim 8, wherein said promoter is a CMV promoter.

10. The cell of claim 8, wherein said cell is a neuroblastoma cell selected from the group consisting of: GOTO, CHP-212, SK-N-BE(2), CHP-243, and SH-SY5Y.

11. The cell of claim 8, wherein said cell is a skin cell carcinoma.

12. The cell of claim 11, wherein said cell is SCC-25.

13. A method of measuring the ability of a compound to effect MCHR1 activity comprising the steps of:
a) providing said compound to the cell of any one of claims 1-12; and
b) measuring MCHR1 activity.

14. The method of claim 13, wherein said step (a) further comprises the presence of an MCHR1 agonist.

15. The method of claim 14, wherein said MCHR1 agonist is human melanin-concentrating hormone.

16. The method of claim 13, wherein said cell is a human neuroblastoma cell.

17. The method of claim 16, wherein said recombinant MCHR1 gene is present in the neuroblastoma cell genome and comprises endogenous nucleic acid encoding for MCHR1 transcriptionally coupled to an exogenous promoter.


SEQUENCE LISTING (9 sequences)

SEQ ID NO: 1 - protein, 353 aa, Human
MDLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGT

SEQ ID NO: 2 - DNA, 1062 bp, Human
atggacctggaagcctcgctgctgcccactggtcccaacgccagcaacacctctgatggccccgataacctcacttcggcaggatcacctcctcgcacggggagcatctcctacatcaacatcatcatgccttcggtgttcggcaccatctgcctcctgggcatcatcgggaactccacggtcatcttcgcggtcgtgaagaagtccaagctgcactggtgcaacaacgtccccgacatcttcatcatcaacctctcggtagtagatctcctctttctcctgggcatgcccttcatgatccaccagctcatgggcaatggggtgtggcactttggggagaccatgtgcaccctcatcacggccatggatgccaatagtcagttcaccagcacctacatcctgaccgccatggccattgaccgctacctggccactgtccaccccatctcttccacgaagttccggaagccctctgtggccaccctggtgatctgcctcctgtgggccctctccttcatcagcatcacccctgtgtggctgtatgccagactcatccccttcccaggaggtgcagtgggctgcggcatacgcctgcccaacccagacactgacctctactggttcaccctgtaccagtttttcctggcctttgccctgccttttgtggtcatcacagccgcatacgtgaggatcctgcagcgcatgacgtcctcagtggcccccgcctcccagcgcagcatccggctgcggacaaagagggtgacccgcacagccatcgccatctgtctggtcttctttgtgtgctgggcaccctactatgtgctacagctgacccagttgtccatcagccgcccgaccctcacctttgtctacttatacaatgcggccatcagcttgggctatgccaacagctgcctcaacccctttgtgtacatcgtgctctgtgagacgttccgcaaacgcttggtcctgtcggtgaagcctgcagcccaggggcagcttcgcgctgtcagcaacgctcagacggctgacgaggagaggacagaaagcaaaggcacctga

SEQ ID NO: 3 - DNA, 8483 bp, Human
  CDS (4208)...(4289)
  CDS (5504)...(6482)
atctctgctaaaattagccgggcatggtggcacatgcttgtaatcccagctactctggaggctgaagccagagaatcacttgaaccaggaggcagaggtttcagtgagctgagatcataccactgcactccagcctgggcgacagagcaagactctgtctcaaatgaaaaaatacatacataaattaattaattaaaaaagaaaggagacagtttattgacaggggagaactctcctgcaacacaggatgtagctggagccagtcctaacatgcttctgggatagctccatgaaatggtttacaataataaggtggggatgctgggatggccattgcagagttttgggattttttgtgcatagtaaaatacacataaaatttgcctcttcactttatatattgtggtaggatatacgtaacataaaatttaccattttaaccacttttaagtgtccaatttagtggcattaagtacgttcacaatgttgtgcagccattatcactgtccatttctagaacttttttttttttaagatggattttcgctattgttgcccaggctggagggtaatggcacaatctcagctcactgcaaccctcacctcccgggttcaagcaattctcctgcctcagcctcctgagtagctgggactacaggcacatgccaccatgcccggctaatttttgtatgcttagtagaggcagggtttcaccatgccagccaggctggtttcaagctcctgacctcaggtgatccgcctgccttggcctcccaaagtgctgggattagaggcaagagtcaccacacctggccagctacacacttttaaacaacaagatctcatgagcactcactcattatcacaaaaacaggaagggggaaatctgttcccatggtccaatcacctcccaccaaacatgcttcctcacttccttaaggacttttctggaagtgcttctcagtgaggccttcctctactgcaaaaagcttcccctgccacaaagagcttctttttttttgagacagagtctcgttctgtcacccaggctggagtgcagtggcgtgatctcagctcactgcaatctccacctcctgggttcaagcgattctcatgcttcagcctcccaagtagctgggattacaggtgcttgccaccacacctggctaatttttgtacatttagtagacacagggtttcaccatgttggccaggctggtctcaaactcctgacctcagtcaatccacccgcctcagcctcccaaaatgttgggattacaggcgtgagccactgtgcccggccaagactgatcctttcaaaacatgtctgatcatgtcttttctcagctgaaaaccccctggtgactcccatttcccctaaaggcaaagccaaagtgcttatactggctggcaaggcctggcagaatctgcccttcctgctccttcctctctgatgacagctcctgctaatctctactatagccacagtgctctccagagtcacactgcacactcctgccacagggcctctgcactgacaatttccatggcagggaatgctggtcctccagaatatttcttcttcttttttttttttttgagacggcgtctcactctgtcacccaggctggagtgcaatggcatgatctcggctcactgcaatttccacctcccgggttcaagcgattctcctgcctcagcctcccaagtagctgggactacaggcgcccgccaccacgcccagctaatttttatatttttaatagagagggggtttcaccttgttggccaggattgtctcgatctcttgacatcatgatccgcctgcctcggcctcccaaagtgctgggatttcaggcgtgagccactgcgcccggccactccagaatatttctaagcatgcttcctcacttccttcaggacttttctcagagaggccttccttgatgatcaatcctctataaaatggtaacctccacctctcttggtattcctgggccctctcaccctgctttaagttttttacagcaattgccaatatcataagttatctttatgtggagtatatattttttcttttttttgagacgtagtctcgctctgtcacccaggctggagtgcagtggcgtggtctcggctcactgcaagctccgcctcccgggttcacgccattctcctgcctcagcctcccgagtagctgggactacaggcacccgccaccacgcccggttaattttttgtattttttaatagagatggggtttcaccatgttagccaggatggtctcgatctcctgacctcgtgatctgccagcctcggcctcccaaagtgctggtattacaggtgtgagccactgcgcccggccatattttttcttttttctgagacagggtctttctgtgtcgcccaggctggagtgcagtggcccaatctcagctcacttcaatctctgcctcctg[450 bp omitted]aattgccaataccatgaattgtcttttgaggggttttctgtctccccgcaacttgaatgcaatcttcatgaggacagggactttatcccccaccccacccccacttttttttcttttttttttactgcaaaacctagaacagtgcttggcaggtagtaagtactcaatgaatgtttgttggatgaacccaataagtaaacaagatagaggacacatgtagggatctgcccgtgaacctcgacccctggctcctgagtctggcagtgggtgcagtggcagctcctgtctgtgagggccccagaggctggcagcaggatgcctccgtggaaaattcccttaacgcctttgcctctgcagctgttcctccgggatgatctctttgggggatcatgctcagatatttgtctcaaagagtcccaggccaaacctcagggacctcagagcgtttagaaaaataacacctctgtgagcttggtccaggcagatcccatgcagagaggagtttgtccccttccagtccccgaggtcctggctattgccagcatggagtgacctgtgtcacctctgagtgccaggcaagggttcagcagctgacgactcagcttctgcaggatgctggcagcatagccagcgagatagttggaagccgtcagggcacagggaaggggccgagggtgccctgagtgtgcatggggggcagccctgctgcagtccaagcctttgattcccaagctatgtgcacagtttcctctggactctgccatgtggcccagccacccatacctggaataggggctaagccaagctgctctctcctccaaagggaggcagcctgtgtgctttgtccgtttgcctttgcagagacctcgatcttcacgcaaggcaagcagcagcccctgtaagcacacgagacaatcccaagtgtcagtgggaaggagatccctttcctgatggggctgcctgtgtccagtccctcccagcttccccagggccctggggctctgcaggcattcagaagtggaagccagccacagcctgggactgaagaggttaatgtgcatctgcctccgaatgttaatgtgtctaggtgatgtcagtgggagccatgaagaagggagtggggagggcagttgggcttggaggcggcagcggctgccaggctacggaggaagacccccttcccaactgcggggcttgcgctccgggacaaggtggcaggcgctggaggctgccgcagcctgcgtgggtggaggggagctcagctcggttgtgggagcaggcgaccggcactggctggatggacctggaagcctcgctgctgcccactggtcccaacgccagcaacacctctgatggccccgataacctcacttcggcaggtgagttgactgggagccctccctcctctgggctgtgggtggaaaatgggaaggtttcacccctgagccaaactgcttgggaaactttatcacagttcttggggacaagatctgtggtctgctttgctctgaggggcaggagaaaagggggcaatggtccgcaggggcagacgggcaggagcagagcagggggcgaaggcatattcagaatggcaaggaaggggggccagccgtgagacagcaggggaaggctcgctgctgggttccaaagatgcttggcagaaaaaattccaggctggaaaagcaagcgagagaagctggagggtggtatgtgggagacagctgggggctcactcctgcactgttagcctcagctttttactcccacttggatgatgaggtctgagacatccttactgccacctgggagaggccctgggaagggaagacttcacagagccatgaggggattaacttttctggtgaattaagcttcctgacatttccagagctgcggtgccctgggattccagctttgaaggagaaaggaaggaaggaaaagaggaaaggcttatgtagataatttttccaggctgctgagctccaacagacagtttctgtctctgcttcactcaagaagcccaggctcagaagataccaatcaaggaaatccccgctaggaagcctggggtagggagagctgctggcttgaccagggcacagccggcaaaagcctctacaagacagtcacccacagatatgcccaagaatcagtacacagtttccaaccagagatctccaaaatgaaacactcagggctacacataggaaaagcacgcacacacacacacacacacatacacagacacttacttttgtgtccttctggctatgctgacgagttttcctggtgaagcccggggctcacagagtaatctctgcagacaactgtggttcttgcctctggtgcctgcaggaggcaggcatgttgtgtccttccaagacagatggctcagggcactctggtaggattcaccaggaaactcatggagaagggaaaagggacaagattagcaacagtgaagggagggagaatggtgggagaggattccagatgaacggtgggtcgctggaggctgagcatgccagcaggatgtcagttctcagagcaaagcccatgtcaaacagccaacgcttgctccttctgtccccaggatcacctcctcgcacggggagcatctcctacatcaacatcatcatgccttcggtgttcggcaccatctgcctcctgggcatcatcgggaactccacggtcatcttcgcggtcgtgaagaagtccaagctgcactggtgcaacaacgtccccgacatcttcatcatcaacctctcggtagtagatctcctctttctcctgggcatgcccttcatgatccaccagctcatgggcaatggggtgtggcactttggggagaccatgtgcaccctcatcacggccatggatgccaatagtcagttcaccagcacctacatcctgaccgccatggccattgaccgctacctggccactgtccaccccatctcttccacgaagttccggaagccctctgtggccaccctggtgatctgcctcctgtgggccctctccttcatcagcatcacccctgtgtggctgtatgccagactcatccccttcccaggaggtgcagtgggctgcggcatacgcctgcccaacccagacactgacctctactggttcaccctgtaccagtttttcctggcctttgccctgccttttgtggtcatcacagccgcatacgtgaggatcctgcagcgcatgacgtcctcagtggcccccgcctcccagcgcagcatccggctgcggacaaagagggtgacccgcacagccatcgccatctgtctggtcttctttgtgtgctgggcaccctactatgtgctacagctgacccagttgtccatcagccgcccgaccctcacctttgtctacttatacaatgcggccatcagcttgggctatgccaacagctgcctcaacccctttgtgtacatcgtgctctgtgagacgttccgcaaacgcttggtcctgtcggtgaagcctgcagcccaggggcagcttcgcgctgtcagcaacgctcagacggctgacgaggagaggacagaaagcaaaggcacctgatacttcccctgccaccctgcacacctccaagtcagggcaccacaacacgccaccgggagagatgctgagaaaaacccaagaccgctcgggaaatgcaggaaggccgggttgtgaggggttgttgcaatgaaataaatacattccatggggctcacacgttgctggggaggcctggagtcaggtttggggttttcagatatcagaaatccccttgggggagcaggatgagacctttggatagaacagaagctgagcaagagaacatgttggtttggataaccggttgcactatatctgtgagctctcaaatgtcttcttcccaaggcaagaggtggaagggtactgactgggtttgtttaaagtcaggcagggctggagtgagcagccagggccatgttgcacaaggcctgagagacgggaaagggcccgatcgctctttcccgcctctcactggtgcgatggaaggtggcctttctcccaagctggtggataatgaaaaataaagcatcccatctctcggcgttccagcatcctgtcaatttcccttttgctctagaggatgcatgtttatttgaggggatgtggcactgagcccacaggagtaaaagcccagtttgctaggaggtctgcttactgaaaacaaggagacctggggtgggtgtggttgggggtcttaaaactaataaaagctggggtcggggggcttttgcagctctggtgacattctctccacggggcacatttgctcagtcactaatccagcttgagtgtccgtgtgttctgcatgtgcaggggtcattctagtgcccggtgtgttggcatcatctttttgctctagcccttcctctccaaaataaaatcaaataaaggaaaatctccacccacatcactctggatgttcttgtggacttgggggtgggtgtgggctggggcggggaaggtgggcagcagaaaagagaaagaggggtcacttggttgtggaatttagatcttggttcatgctgcatttttaggaagcatgaagcagggatctgtttttctgaacccacagggaggattcagtggcataaatggaattactggggattcattagattttgcagttctgctgctgggcttgttcttgggactcagcttcctgtcttctgcacaaaatcccctgggctttgttgtcatcagtgatgagtcctcaggccccaagtcccaacccccactcccccgcctcaaccctcacccccgctgagtcaccagccgcagagccagctcttagggcagctgaagcctctctgctttctacagctgagatctggtgtgggcaccttgaacagaagattacagccggggccactgggaggcagccacgactgctgcttggctgctgcttcttggtgtcttcactgagaggggactgggagccgtcagtgcagtgctcagcagaccttactgagagagcgggagaaagctgcaagcatcctgaaagcaggggcagcagcacagcctgttcctcttcagagctgcagcagggagcctcttcagaaaacttctgggcagcttccttgggtcctggggactttttttttgagacagagtatcgctctgtcgcccaggctgaagtgcagtggtgtgatctctgctcactgcaagctccgcctcccgggttcacaccattctcctgcctcagcctcccgagtagctgggactgcaggcatccgccaccacgcccggctaatttttgtatttttagtaaagacggggtttcaccatgttagccaggatggtctcgatctcctaacctcatgatctgcctgcctcggcctcccaaggtgctgagactgcaggcgtaagccaccgtgcccggcttttttttttttttttttttttggacacagggccttgccctgttgcccaggctgtagtgcaggtgacatgatcacagctcactgcagcctcgacctcccagattcaagcaatcctcccacctcagcctcccaagtagctgg

SEQ ID NO: 4 - DNA, 24 bp, Artificial Sequence
  Oligonucleotide Primer
atggacctggaagcctcgctgctg

SEQ ID NO: 5 - DNA, 21 bp, Artificial Sequence
  Oligonucleotide Primer
gccagcaacacctctgatggc

SEQ ID NO: 6 - DNA, 23 bp, Artificial Sequence
  Oligonucleotide Primer
ggccccgataacctcacttcggc

SEQ ID NO: 7 - DNA, 22 bp, Artificial Sequence
  Oligonucleotide Primer
gaggagatctactaccgagagg

SEQ ID NO: 8 - DNA, 22 bp, Artificial Sequence
  Oligonucleotide Primer
gcccatgagctggtggatcatg

SEQ ID NO: 9 - DNA, 24 bp, Artificial Sequence
  Oligonucleotide Primer
gtggacagtggccaggtagcggtc